(19) United States
(12) Patent Application Publication
(10) Pub. No.: US 2015/0305946 A1
(43) Pub. Date: Oct. 29, 2015

(54) Rolled Absorbent Hygiene Device

(71) Applicant: Yvonne L. Henderson, Morrisville, PA (US)

(72) Inventor: Yvonne L. Henderson, Morrisville, PA (US)

(21) Appl. No.: 14/689,003

(22) Filed: Apr. 16, 2015

Related U.S. Application Data
(60) Provisional application No. 61/980,266, filed on Apr. 16, 2014.

(51) Int. Cl. A61F 13/45; A61F 13/53

(52) U.S. Cl. CPC A61F 13/45; A61F 13/53; A61F 2013/530029

(57) ABSTRACT

An elongated, absorbent device is provided for absorbing moisture and discharge between a user's legs while sleeping. The absorbent device comprises a rolled absorbent tissue paper formed into a cylindrical, multilayer device that can be inserted between a user's legs and between a user's buttocks while sleeping. The device is secured in a roll form using an absorbent string material, while the length and diameter of the device are suited for being placed between a user's legs. The material is absorbent and compressible, whereby the body of the user and the user's undergarment secure the device in place to absorb moisture. In a preferred embodiment, the device is formed using a folded sheet of material that is then rolled and secured into a rolled form using the absorbent string material. The device is discarded after use.

### CROSS REFERENCE TO RELATED APPLICATION

This application claims the benefit of U.S. Provisional Application No. 61/980,266 filed on Apr. 16, 2014. The above identified patent application is herein incorporated by reference in its entirety to provide continuity of disclosure.

### BACKGROUND OF THE INVENTION

### Field of the Invention

The present invention relates to absorbent hygiene devices. More specifically, the present invention pertains to a rolled absorbent device for absorbing discharge from a user while sleeping. The device is formed using a rolled absorbent material and secured in a rolled form by absorbent string, whereby the size and shape allows the device to be placed between the legs and between the buttocks of a wearer while sleeping.

During menstruation, heavy bleeding can occur and lead to soiled clothes and bedding. Typical solutions to prevent soiled clothing and unwanted leakage include tampon devices and sanitary pads. However, during periods of sleep when a woman may turn on her side or sleep in various positions, these typical devices may not be suitable for preventing fluid from leaking completely. The result of such uncontrolled leakage is soiled linens and clothing requiring replacement. This may also lead to uncomfortable sleeping positions to prevent such leakage. Conventional pads or sanitary napkins may not fully prevent leaks from occurring, and may not be effective during heavy bleeding.

The present invention relates to a rolled sanitary device that is adapted to be placed between a user's legs or between a user's buttocks to capture and absorb leaking moisture. The device is ideally suited for use while sleeping, whereby the rolled device is placed between the buttocks and legs of the sleeping user, and the user's undergarments maintain the position of the absorbent device while sleeping. The device is formed into a cylindrical roll form, whereby the absorbent material includes several layers that are layered on top of one another through the diameter of the device. An absorbent string material maintains the shape of the roll. The rolled form improves moisture absorption and moisture capture within the device over existing absorbent device with a homogeneous cross section.

### SUMMARY OF THE INVENTION

The following summary is intended solely for the benefit of the reader and is not intended to be limiting in any way. The present invention provides a new absorbent hygiene device that can be utilized for providing convenience for the user when absorbing moisture and discharge during menstruation or for otherwise capturing discharge or fluid in the crotch and buttocks of a user.

It is therefore an object of the present invention to provide a new and improved absorbent hygiene device that has all of the advantages of the typical hygiene pads, while providing a construction that is suited for use between the legs or buttocks of a sleeping user and for trapping moisture therein.

It is another object of the present invention to provide an absorbent hygiene device that is a rolled, cylindrical device having multiple layers through its cross section and secured in a roll form using string.

Another object of the present invention is to provide an absorbent hygiene device in which the string supporting the roll in its rolled form is absorbent itself, assisting the absorbent material in capturing and trapping moisture from a sleeping user.

Yet another object of the present invention is to provide an absorbent hygiene device that is adapted to be placed between the legs or between the buttocks of a wearer and held in place by the undergarments of the wearer.

Another object of the present invention is to provide an absorbent hygiene device that includes multiple layers that absorb and trap moisture therein, whereby the layered construction ensures moisture is trapped within certain layers and the device useful for a period of hours while the wearer is sleeping.

Another object of the present invention is to provide an absorbent hygiene device that can be provided in rolled form or in the form of a kit, whereby the user can determine the appropriate thickness of the rolled device for her exact body type and comfort level.

Other objects, features and advantages of the present invention will become apparent from the following detailed description taken in conjunction with the accompanying drawings.

### BRIEF DESCRIPTIONS OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows a perspective view of the absorbent hygiene device of the present invention.

FIG. 2 shows a cross sectional end view of the absorbent hygiene device of the present invention.

FIG. 3 shows a view of the device in a working state.

FIG. 4 shows the steps in which the kit of the present invention can be used to create the absorbent hygiene device of the present invention.

### DETAILED DESCRIPTION OF THE INVENTION

Reference is made herein to the attached drawings. Like reference numerals are used throughout the drawings to depict like or similar elements of the absorbent hygiene device of the present invention. For the purposes of presenting a brief and clear description of the present invention, the preferred embodiment will be discussed as used for absorbing and capturing fluid during menstruation and while a user is sleeping, using a device that is placed between the legs and/or between the buttocks of a user. The figures are intended for representative purposes only and should not be considered to be limiting in any respect.

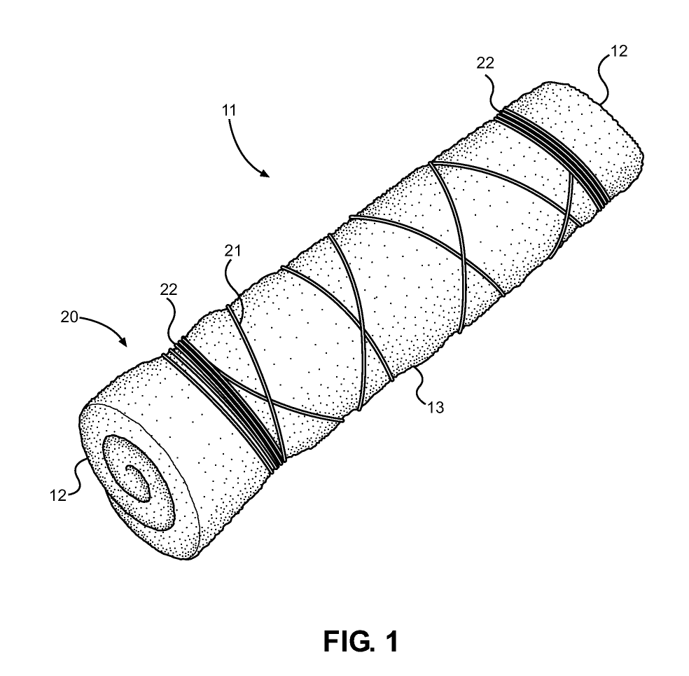

Referring now to FIG. 1, there is shown a perspective view of the absorbing member 11 of the present invention. The absorbing member 11 comprises an elongated, cylindrical device that is used to absorb and capture moisture, discharge, and fluid from a user while the user is sleeping or otherwise stationary. The device has a length and a diameter that is suited for placement between a user's legs and/or between the user's buttocks for absorbing fluid. The primary purpose of the device is to absorb menstrual fluid while a user is sleeping; however, other alternative uses are contemplated beyond menstruation fluid absorption and use while sleeping.

The device is one that is provided to a user either as a kit of material, or as a formed device that can be unwrapped and utilized without any preparation. The device itself, either prepared by the user or prepackaged, comprises a rolled assembly having multiple layers through its cross section. The layers are one or more sheets of absorbent material that are aligned, folded, and then rolled together into the cylindrical shape depicted in FIG. 1. To retain the rolled, cylindrical shape, absorbent string 20 is used to wrap around the device, thereby ensuring the device maintains its shape while in use. The absorbent string 20 comprises a cotton string material or equivalent absorbing material string, whereby the string functions as a structural member and an additional absorbing element in the assembly.

Menstrual fluid can be handled using a sanitary pad or tampon device; however, during periods of heavy bleeding, fluid can bypass these measures and cause soilage of sheets and of undergarments while sleeping. The user may also have alternative discharges or fluid leakage not related to menstruation that can cause similar soiling of undergarments and of bedding material. The present invention is one that is adapted to be placed between the legs or between the buttocks of a stationary user to absorb leaking fluid. The device is placed between the user's skin and an undergarment, whereby the undergarment maintains the position of the device and the device is sized to fit into these areas without undue discomfort for the user. The device absorbs and retains fluid, whereafter the device is discarded.

In its ready form, the absorbent member 11 comprises a rolled, cylindrical shape with a first and second end 12, a cylindrical body 13, and a multilayer cross section. The multilayer, rolled cross section facilitates several absorbing layers and capture of fluid within the device. As outer layers are saturated, inner layers absorb moisture and draw the moisture towards the center of the device. The discontinuities between the layers also capture the fluid and prevent the fluid from leaking once absorbed. Finally, the string material 20 is used to retain the rolled shape of the device. The string material is an additional absorbent material that is first wrapped around one end 22 of the device, and then wrapped around the cylindrical body 13 of the device until the string is wound around the second end 22. The string supports the device 11, and increases overall absorption of fluid.

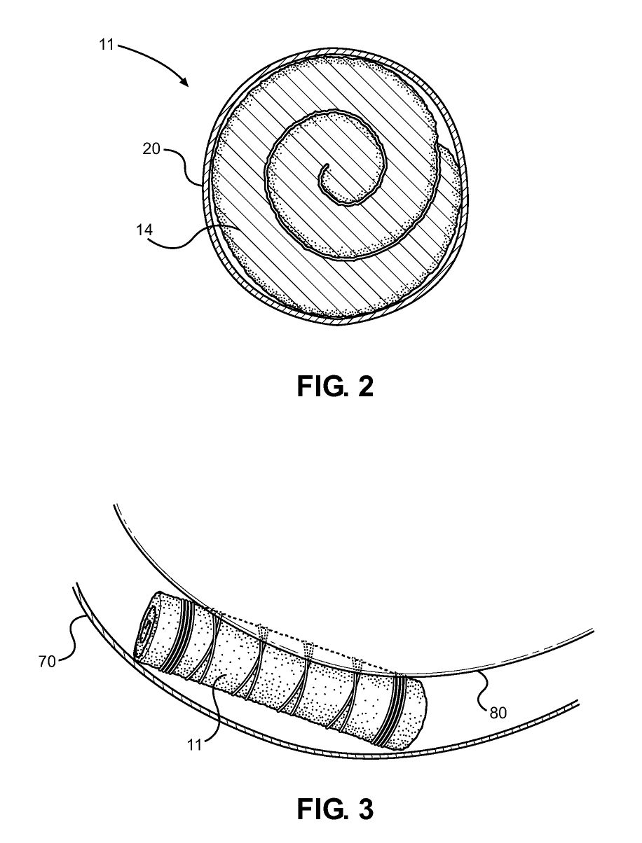

Referring to FIG. 2, there is shown a cross section view of the absorbing member 11 of the present invention. The absorbent member 11 comprises a rolled construction with several layers 14. The layers 14 are formed as a result of the absorbent material being rolled, as well as the construction being composed of one or more absorbent sheets of material. The sheets of absorbent material are preferably a tissue material or cotton material that absorbs moisture. The material may break down when flushed, but is suitable for placement in a highly moist environment while in use without breaking down. The material absorbs and traps the moisture within its cross section, while the string material 20 retains the shape of the member 11 and adds an additional absorbing element. The string material 20 absorbs moisture and retains the moisture therein similarly without breaking down the material. Contemplated materials include cotton string or equivalent.

Referring to FIG. 3, there is shown a view of the absorbing member 11 in a working state. The absorbing member 11 comprises a cylindrical shape with dimensions suitable for placement between a user's legs or between a user's buttocks 80, as showing FIG. 3. Exemplary dimensions contemplated are 2½ inches in length and ½ inches in diameter. However, this is merely an example of suitable dimensions, falling within the scope of the claims. In use, the user's undergarment 70 presses the absorbent member 11 against the user's skin and between the user's legs or between the user's buttocks 80. In this position, the user can sleep on her side, back, or stomach, while the member 11 remains in position to absorb moisture that may leak during menstruation or from other fluid sources. The absorbing member 11 is designed for external use, either between the user's legs or between the buttocks of the user and exterior to the body of the user.

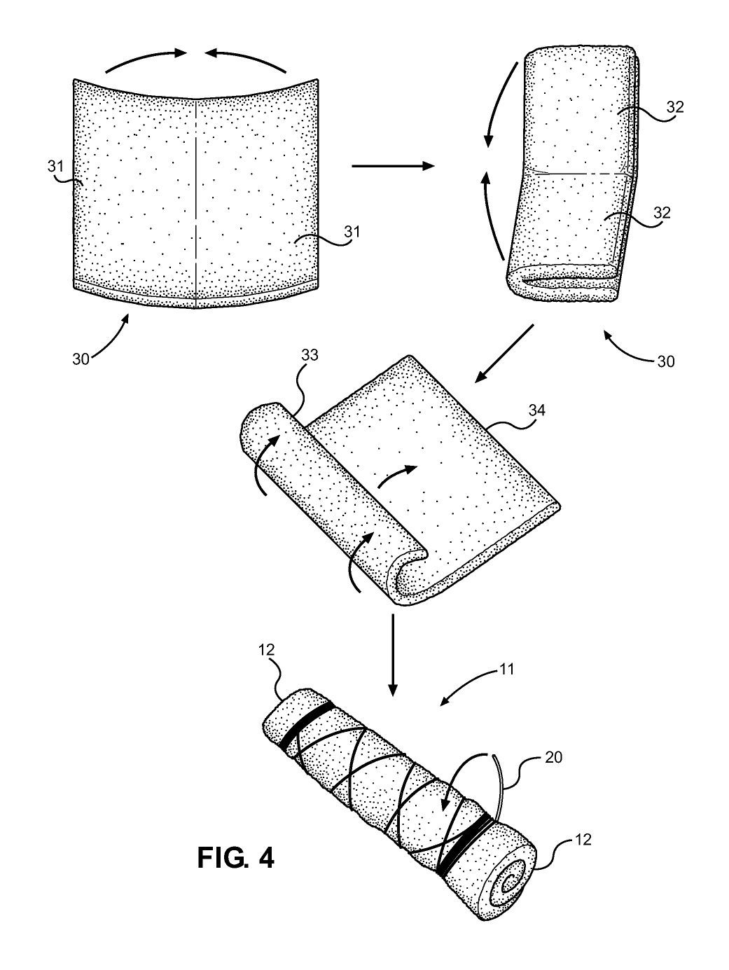

Referring to FIG. 4, there is shown a view of the kit provided to users and the method of creating the absorbing member 11 of the present invention. The kit comprises a plurality of sheets 30 of absorbing material that are rectangular in shape. One or more sheets 30 are placed on top of one another and the edges are folded together such that the halves 31 of the sheets are pressed together. Thereafter, the sheets can be folded again, whereby the quarters 32 of the sheets are folded together. Finally, the folded material is rolled from one end 33 to the opposite end 34 to create the rolled shape. The thickness of the sheets 30 will determine how many times to fold the material prior to rolling the same. Once in a rolled form, the user wraps the absorbent string 20 therearound, which is also provided in the kit. Several lengths of string 20 are provided to create several absorbing members using the plurality of sheets 30. Once rolled and secured by the string 20, the absorbing member has a cylindrical shape with a pair of ends 12 and a multilayer cross section. The member 11 can then be placed between a user's legs or buttocks and covered by an undergarment for absorbing moisture from a user.

It is submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1. An absorbent feminine hygiene device, comprising:
an absorbing member having a cylindrical shape with an axial length and a diameter;
the absorbing member formed from one or more absorbent tissue sheets rolled into the cylindrical shape;
wherein the absorbent sheets form multiple layers of tissue sheets through the diameter of the absorbing member;
the absorbing member having an outer surface and a pair of ends;
an absorbent string wrapped around the outer surface of the absorbing member, the absorbent string wrapping around the outer surface between the ends of the absorbing member;
whereby the length and the diameter of the absorbing member is adapted to be positionable between the buttocks of a wearer and supported by an undergarment, whereby the absorbing member and the absorbent string are adapted to absorb fluid from the wearer.

2. The absorbent feminine hygiene device of claim 1, wherein the absorbent string comprises a cotton string material.

3. A kit for creating one or more absorbent feminine hygiene devices, comprising:
a packaging supporting:
a plurality of rectangular absorbent sheets; and
a plurality of elongated lengths of absorbent string;
instructions for folding one or more of the absorbent sheets one or more times and thereafter rolling the one or more of the absorbent sheets before securing the one or more of the absorbent sheets into a roll form using one of the absorbent string.
